(19) United States
(12) Patent Application Publication
(10) Pub. No.: US 2019/0217032 A1
(43) Pub. Date: Jul. 18, 2019

(54) COUGH MASK ASSEMBLY

(71) Applicant: Neda Shariff, Lake Tapps, WA (US)

(72) Inventor: Neda Shariff, Lake Tapps, WA (US)

(21) Appl. No.: 15/873,423

(22) Filed: Jan. 17, 2018

(51) Int. Cl. A61M 16/04; A41D 13/11

(52) U.S. Cl. CPC A61M 16/047; A61M 16/0605; A41D 13/11

(57) ABSTRACT

A cough mask assembly for inhibiting the spread of microbes through coughing includes a cover. A strap is attached to the cover to and secures the cover around the neck of a person. A panel is attached to the cover and conforms a shape of the cover to a face of the person.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to cough suppressant devices and more particularly pertains to a new cough suppressant device for inhibiting the spread of microbes through coughing.

### BRIEF SUMMARY OF THE INVENTION

An embodiment of the disclosure meets the needs presented above by generally comprising a cover. A strap is attached to the cover and is configured to secure the cover around the neck of a person. A panel is attached to the cover and is configured to conform a shape of the cover to a face of the person.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top side view of a cough mask assembly according to an embodiment of the disclosure.

FIG. 2 is a front view of an embodiment of the disclosure.

FIG. 3 is a back view of an embodiment of the disclosure.

FIG. 4 is a cross-sectional view of an embodiment of the disclosure taken along line 4-4 of FIG. 2.

FIG. 5 is a top view of an embodiment of the disclosure.

FIG. 6 is a front side view of an embodiment of the disclosure.

### DETAILED DESCRIPTION OF THE INVENTION

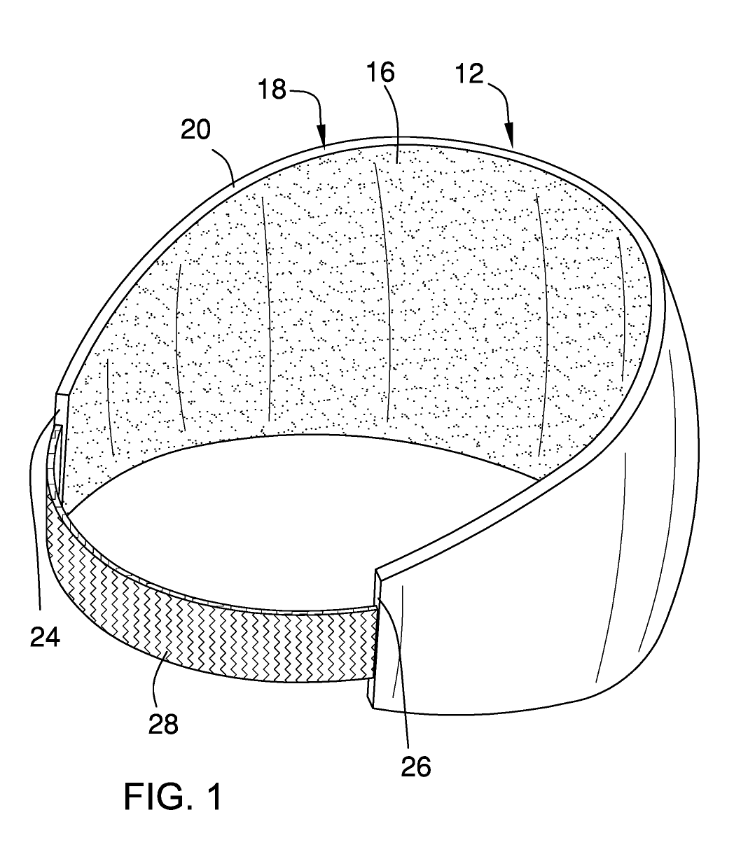

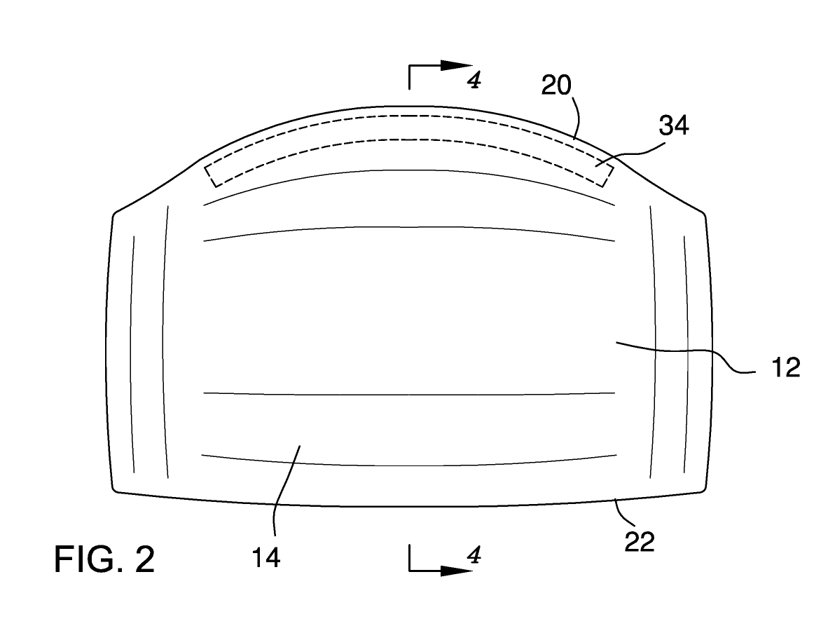

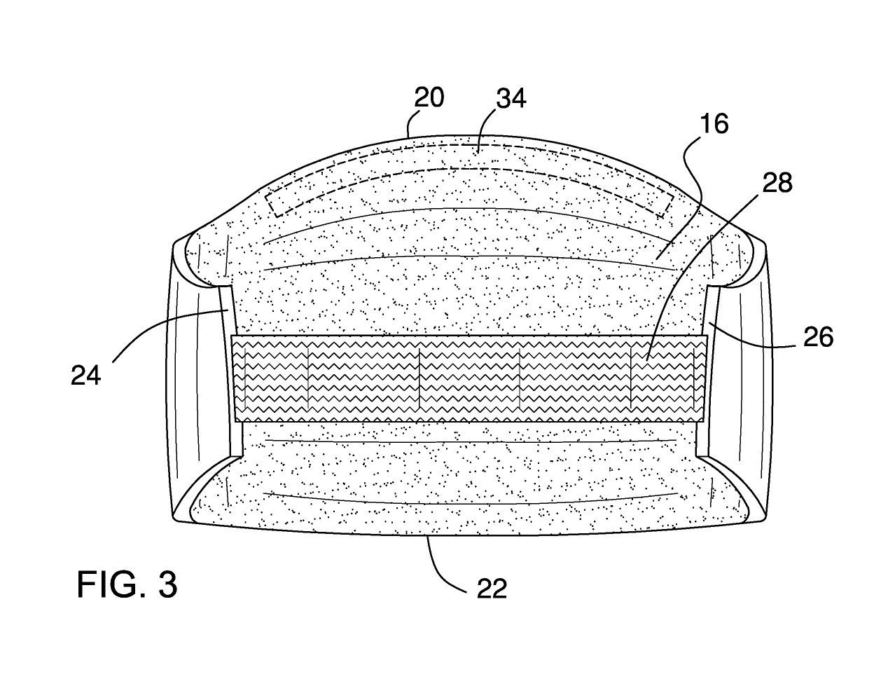

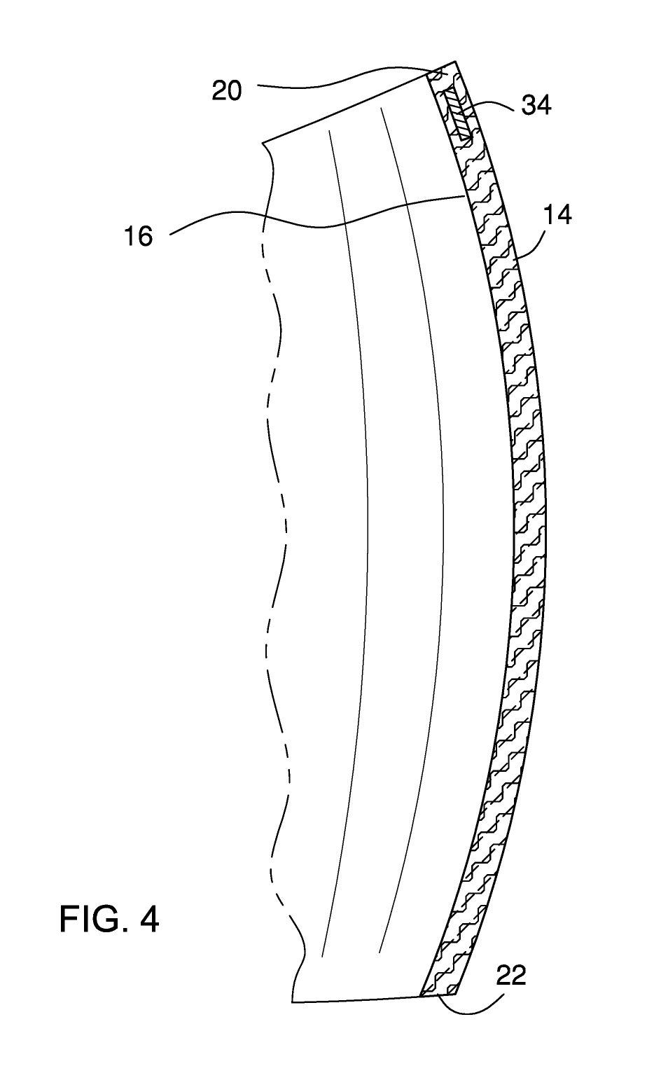

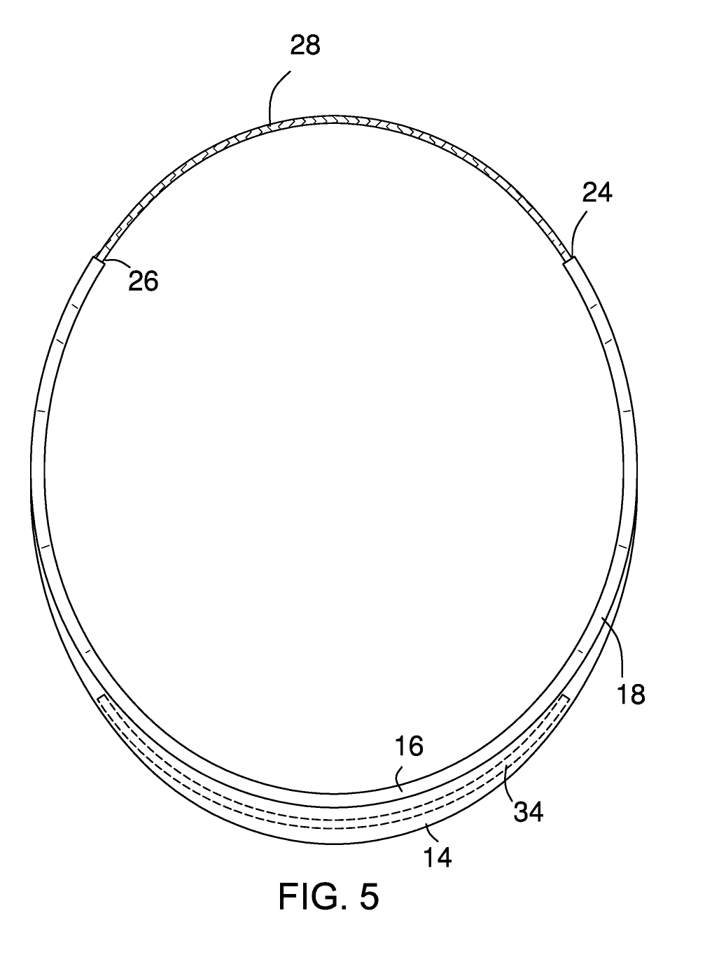

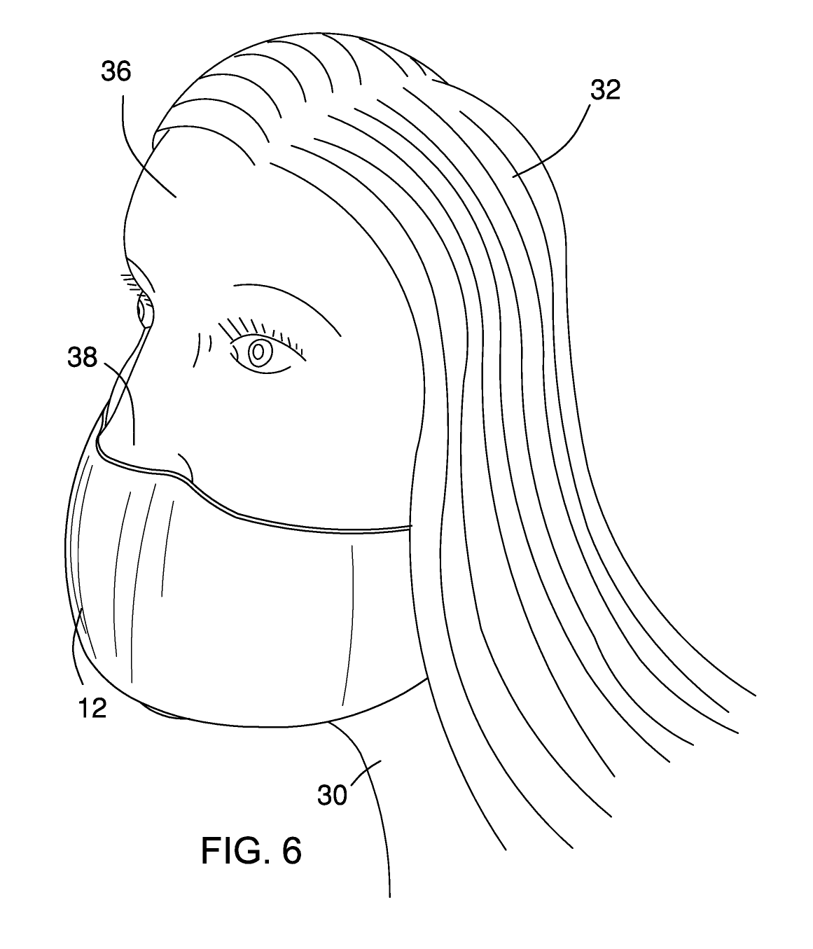

With reference now to the drawings, and in particular to FIGS. 1 through 6 thereof, a new cough suppressant device embodying the principles and concepts of an embodiment of the disclosure and generally designated by the reference numeral 10 will be described.

As best illustrated in FIGS. 1 through 6, the cough mask assembly 10 generally comprises a cover 12 that has a forward side 14, a rear side 16 and a peripheral edge 18 that extends between and is attached to the forward side 14 and the rear side 16. The peripheral edge 18 has a top side 20, a bottom side 22, a first lateral side 24 and a second lateral side 26.

A strap 28 is attached to the cover 2 and extends between the first lateral side 24 and the second lateral side 26. The strap 28 is comprised of a resiliently stretchable material and secures the cover 12 around a neck 30 of a person 32.

A panel 34 is attached to the cover 12 and conforms a shape of the cover 12 to a face 36 of the person 32. The panel 34 is positioned within the cover 12 and is parallel to and extends adjacently along the top side 20 of the peripheral edge 18. The panel 34 is comprised of a bendable and shape retaining material. The panel 34 is bendable into a shape to be contoured to a shape of a person's nose 38 to retain the cover 12 in abutment with the face 36 of the person. The rear side 16 of the cover 12 abuts the person's face 36 when the panel 34 is secured to the person's nose 38 wherein the person 32 coughs into the rear side 16 of the cover 12.

In use, the cover 12 is wearable around the person's neck 30 wherein with the strap 28 secure the cover 12 around the person's neck 30. The panel 34 is bent to conform to the shape of the person's face 36. The panel 34 is positioned on the person's nose 38 to secure the cover 12 to the person's face 36. The rear side 16 of the cover 12 catches a cough of the person 32 to inhibit the spread of microbes.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A mouth covering assembly:
a cover;
a strap being attached to said cover, said strap being configured to secure said cover around a neck of a person; and
a panel being attached to said cover and being configured to conform a shape of said cover to a face of the person.

2. A mouth covering according to claim 1, wherein said cover has a forward side, a rear side and a peripheral edge extending between and being attached to said forward side and said rear side, said peripheral edge having a top side, a bottom side, a first lateral side and a second lateral side.

3. A mouth covering according to claim 2, wherein said strap extends between said first lateral side and said second lateral side of said cover.

4. A mouth covering according to claim 3, wherein strap is comprised of a resiliently stretchable material.

5. A mouth covering according to claim 4, wherein said panel is positioned within said cover and being parallel to and extending adjacently along said top side of said peripheral edge.

6. A mouth covering according to claim 5, wherein said panel is comprised of a bendable and shape retaining material, said panel being bendable into a shape to be configured to be contoured to a shape of a person's nose to retain said cover in abutment with a face of the person.

7. A mouth covering according to claim 6, wherein said rear side of said cover abuts the person's face when said panel is secured to the person's nose wherein the person coughs into said rear side of said cover.

8. A mouth covering assembly:
a cover having a forward side, a rear side and a peripheral edge extending between and being attached to said forward side and said rear side, said peripheral edge having a top side, a bottom side, a first lateral side and a second lateral side;
a strap being attached to said cover, said strap extending between said first lateral side and said second lateral side, said strap being comprised of a resiliently stretchable material, said strap being configured to secure said cover around a neck of a person; and
a panel being attached to said cover and being configured to conform a shape of said cover to a face of the person, said panel being positioned within said cover and being parallel to and extending adjacently along said top side of said peripheral edge, said panel being comprised of a bendable and shape retaining material, said panel being bendable into a shape to be configured to be contoured to a shape of a person's nose to retain said cover in abutment with a face of the person, said rear side of said cover abutting the person's face when said panel is secured to the person's nose wherein the person coughs into said rear side of said cover.
